(19) United States
(12) Patent Application Publication
(10) Pub. No.: US 2017/0354177 A1
(43) Pub. Date: Dec. 14, 2017

(54) ANTIFUNGAL COMPOSITION COMPRISING ANTIFUNGAL PEPTIDE AND TERPENE ALCOHOL

(71) Applicants: MARUHA NICHIRO CORPORATION, Tokyo (JP); TEIKYO UNIVERSITY, Tokyo (JP)

(72) Inventors: Keishi IOHARA, Ibaraki (JP); Makoto MITARAI, Tokyo (JP); Shigeru ABE, Tokyo (JP); Kazumi HAYAMA, Tokyo (JP)

(73) Assignees: MARUHA NICHIRO CORPORATION, Tokyo (JP); TEIKYO UNIVERSITY, Tokyo (JP)

(21) Appl. No.: 15/510,432

(22) PCT Filed: Sep. 1, 2015
(86) PCT No.: PCT/JP2015/074885

§ 371 Date: Mar. 10, 2017

(30) Foreign Application Priority Data
Sep. 12, 2014 (JP) 2014-186288

(51) Int. Cl. A23L 33/17; A61K 38/01; A61K 31/122; A61K 31/045; A61Q 19/00; C07K 1/00

(52) U.S. Cl. CPC A23L 33/17; A61K 31/122; A61K 31/045; A61K 38/01; C07K 1/00; A61Q 19/00

(57) ABSTRACT

A composition with an enhanced antifungal activity, which comprises peptides as a protamine hydrolysate and which is suitable for industrial application at a low cost, can be provided, by using a combination of a protamine hydrolysate and a terpene alcohol as an active ingredient of a composition having an antifungal activity.

### TECHNICAL FIELD

The present invention relates to a composition having an antifungal activity comprising a combination of a protamine hydrolysate and a terpene alcohol. The composition having an antifungal activity can be widely used in functional foods, cosmetics, drugs, quasi-drugs, etc., for prevention or treatment of fungal infection diseases.

### BACKGROUND ART

Yeasts and filamentous fungi are eukaryotes and called a fungus in contrast to bacteria as prokaryotes. Some species of fungi exhibit pathogenicity to humans, which are causative microorganisms causing fungal infection. Among fungi, fungi of genus Candida grow widely in human digestive tracts and usually do not exhibit pathogenicity. However, it is known that the Candida fungi cause various diseases when host defense function becomes lower. Particularly, oral, pharyngeal and esophagus candidiasises are diseases occurring commonly due to reduced secretion of saliva, diabetes mellitus, chronic administration of antibiotics or immunosuppressive agents, decrease of mouth hygiene management, etc.

As the antifungal agent used for treatment of mycosises as an internal antifungal agent, amphotericin B as a polyene type agent, flucytosine as a fluoropyridine type agent, miconazole as an imidazole type (azole type) agent, fluconazole as a triazole type (azole type) agent, itraconazole as an azole type agent, etc. are conventionally used in clinical practice. The kind of the disease-causing fungi targeted by these antifungal agents and the drug-susceptibility of each of the disease-causing fungi vary depending on each antifungal agent. The representative fungi targeted by these antifungal agents include, for example, the genus Candida, the genus Cryptococcus, the genus Aspergillus, the genus Trichophyton, the genus Malassezia, the genus Coccidioides, etc. However, in treatments using these antifungal agents, though infections by fungi are cured temporarily, recurrence is observed in most cases and a drug-resistant strain appears in long-term use. Thus, these problems generate a requirement of introduction of novel treatment methods. Further, oral candidiasises found in aged people, especially in denture wearers, are diseases recurring even if a temporal therapeutic effect is observed as long as the reason for increased susceptibility is not removed. It has been required to develop novel prophylactic means such as a mouthwash, a functional food, etc. as an alternative to the antifungal agent (Non-Patent Literature 1).

In contrast, protamine is a basic protein having an antibacterial activity obtained from testes of fish (milt) and is a food preservative derived from natural products (Patent Literature 1). The milt as a raw material of protamine has traditional eating history and has been eaten as an ingredient in hot pot cooking. Thus, an acute toxicity test for mice and a subchronic toxicity test for rats using the milt has been conducted (Non-Patent Literature 2). Since its safeness has been confirmed, protamine is an antibacterial agent excellent in safety aspect (Non-Patent Literature 3). It is known that protamine shows an especially strong antibacterial activity on gram-positive bacteria such as the genus Bacillus, Lactobacillus, etc., among microorganisms (Non-Patent Literature 4), but protamine shows a very weak antifungal activity to yeasts and filamentous fungi (Non-Patent Literature 5).

A method of hydrolyzing protamine, for enhancing the antifungal activity of protamine, is known, and it has been reported that the following peptides (1) to (3) or salts thereof contained in the hydrolysate have an antifungal activity (Patent Literature 1).

(1) A peptic consisting of an amino-acid sequence of SEQ ID No. 1 represented by Ile Arg Arg Arg Arg Pro Arg Arg, or a salt thereof.

(2) A peptic consisting of an amino-acid sequence of SEQ ID No. 2 represented by Ser Arg Arg Arg Arg Arg Arg Gly Gly Arg Arg Arg Arg, or a salt thereof.

(3) A peptic consisting of an amino-acid sequence of SEQ ID No. 3 represented by Val Ser Arg Arg Arg Arg Arg Arg Gly Gly Arg Arg Arg Arg, or a salt thereof.

On the other hand, it was recognized that a peptide containing a specific amino-acid sequence obtained by hydrolyzing protamine exhibited a high antibacterial activity in vitro, and, however, a tendency of lowering of the activity especially under high salt concentration in vivo (Non-Patent Literature 1). Then, a method of synthesizing a peptide for circularization to enhance its antibacterial activity in vivo, has been reported (Non-Patent Literature 6).

On the other hand, it is known that certain kinds of plant essential oils or essential oil components enhance skin penetration of other drugs. It has been reported that if an antifungal agent was used in combination with an essential oil or a terpene alcohol having an antifungal activity for enhancing the therapeutic effect of conventional antifungal agents, the skin permeability of a drug increased and its blood concentration was enhanced remarkably (Patent Literature 2). The terpene alcohol here referred to denotes a mono-terpene alcohol having 10 carbon atoms in one molecule and a sesqui-terpene alcohol having 15 carbon atoms in one molecule. For example, it is known that each of both palmarosa oil and its major constituent, geraniol, remarkably enhance the blood concentration of terbinafine, an antifungal agent administered to skin. It is known that each of lavender oil and its major constituent, linalool; patchouli oil and its major constituent, patchouli alcohol; and lindera umbellata oil and its accessory constituent, nerolidol, has the same effect, though its degree varies (Patent Literature 2).

Candidiasises on skin and mucosal membrane, particularly oral candidiasis in aged people and vaginal candidiasis in females each exhibit very high incidence frequency. In general, they are treated by bringing an antifungal agent into direct contact with an affected area, however, they are intractable and tend to repeat recurrence. As a result of use of the drug for a long period of time for suppressing recurrence, side effects such as hepatic function disorders occur and a risk of emergence of a drug-resistant strain increases.

### REFERENCE LIST

### Patent Literature

Patent Literature 1: Japanese Patent No. 4520477
Patent Literature 2: JP-A No. 2010-126532

### Non-Patent Literature

Non-Patent Literature 1: Abe shigeru: Development of mouse oral candidiasis model and application thereof, Fungus journal, 45, 227-231 (2004)
Non-Patent Literature 2: Tada yukie, el al.: Subchronic toxicity test by rat of natural preservative milt protein, Tokyo Metropolitan Institute of Public Health, Annual report of research, 49, 267-276 (1998)
Non-Patent Literature 3: lohara keiji, Nemoto erika: Use of milt protein (protamine) for food, Monthly publication Food Chemical, April issue, 24-32 (2006)
Non-Patent Literature 4: Bulletin of the Japanese Society of Scientific Fisheries, 50, 1715 (1984)
Non-Patent Literature 5: Bulletin of the Japanese Society of Scientific Fisheries, 52, 1061 (1986)
Non-Patent Literature 6: 57-th The Japanese Society for Medical Mycology, General meeting-Academic meeting Abstracts collection, 98 (2013)

### SUMMARY OF THE INVENTION

### Problem to be Solved by the Invention

A treatment utilizing a peptide obtained by hydrolyzing protamine and an essential oil ingredient has a merit that side effects and a risk of emergence of a drug-resistant strain are low. However, in general, such naturally occurring ingredients have a lower antifungal activity in some cases than antifungal agents having a chemically synthesized non-peptide compound as an active ingredient. Thus, it is necessary to further enhance the antifungal activity in such above cases. Further, cyclization of a peptide for securing an in vivo activity under high salt concentration as disclosed in Non-Patent Literature 6 was not suitable for industrial application of an antifungal agent at a low cost because of a high cost for synthesis of a cyclized peptide.

An object of the present invention is to provide a composition comprising as an active ingredient a protamine hydrolysate and having an antifungal activity, the composition showing a further improved antifungal activity and being suitable for industrial application at a low cost.

### Means for Solving the Problem

The composition having an antifungal activity (antifungal composition) according to the present invention is characterized in comprising a protamine hydrolysate and a terpene alcohol as an active ingredient.

The present invention further includes the following embodiments.

(1) An antifungal agent comprising the antifungal composition described above.

(2) A food product containing the antifungal composition described above.

(3) A pharmaceutical product, or a drug, or a quasi-pharmaceutical product, or a quasi-drug comprising the antifungal composition described above.

(4) A cosmetic containing the antifungal composition described above.

(5) A method of using the antifungal composition described above as an active ingredient for obtaining an antifungal activity in production of a food product or a cosmetic.

(6) A method of using the antifungal composition described above as an active ingredient for obtaining an antifungal activity in production of a pharmaceutical product or a quasi-pharmaceutical product.

### Effect of the Invention

The present invention can provide a composition not only having an antifungal activity enhanced remarkably by a synergistic effect of a protamine hydrolysate obtained by hydrolysis of protamine with a terpene alcohol, but also having extremely high safeness without problems such as emergence of a drug-resistant strain and a side effect. The intended applications of the antifungal under consideration include oral care materials having an antifungal effect (oral rinse, toothpaste, denture cleaning agent, etc.), pharmaceutical products for fungal infection diseases such as bedsore, vaginal candidiasis, esophageal candidiasis, etc. (antifungal agent), quasi-pharmaceutical products (mouthwash, mouth cleaning agent, medical tooth powder, etc.) and cosmetics (ointment, liquid, suspension, emulsion, aerosol, foam, granule, powder, tablet, capsule, etc.). Oral candidiasis is a symptom occurring particularly in aged people and dry mouth patients, and needs thereof in the further aging society are large. Further, it is possible to apply the present invention not only to oral candidiasis but also to other fungal infection diseases such as candidiasises like bedsore, vaginal candidiasis, esophageal candidiasis, etc., Malasseziosis, Cryptococcus infection, etc, thereby it is possible to improve and treat pathological conditions and to prevent infection. Since protamine is described in a list of existing additives for food preservatives, it is possible to use protamine not only in pharmaceutical products and quasi-pharmaceutical products but also in mouthwashes and functional foods such as healthy food products or food products for specified health use, and there is a merit that protamine is easily available and easy to use for patients.

### BRIEF EXPLANATION OF DRAWINGS

FIG. 1 is a graph showing the relative value of an antibacterial activity in in vitro evaluation when a protamine hydrolysate and Hinokitiol are used in combination.

FIG. 2 is a graph showing the relative value of an antibacterial activity in in vitro evaluation when a protamine hydrolysate and terpinen-4-ol are used in combination.

FIG. 3 is a graph showing the relative value of an antibacterial activity in in vitro evaluation when a protamine hydrolysate and geraniol are used in combination.

FIG. 4 is a graph showing the relative value of an antibacterial activity in in vitro evaluation when a protamine hydrolysate and menthol are used in combination.

FIG. 5 is a graph showing the relative value of an antibacterial activity in in vitro evaluation when a protamine hydrolysate and cinnamaldehyde are used in combination.

FIG. 6 is a graph showing the relative value of an antibacterial activity in in vitro evaluation when a protamine hydrolysate and capric acid are used in combination.

FIG. 7 is a graph showing the relative value of an antibacterial activity in in vitro evaluation when a protamine hydrolysate is used alone.

FIG. 8 is a view showing the value of tongue score in in vivo evaluation when a protamine hydrolysate and Hinokitiol are used in combination (*: p<0.05 according to Steel Dwass method).

FIG. 9 is a graph showing the results in in vitro evaluation when a protamine hydrolysate and Hinokitiol are used in combination.

FIG. 10 is a graph showing the results in in vitro evaluation when a protamine hydrolysate and Hinokitiol are used in combination.

FIG. 11 is a graph showing the results in in vitro evaluation of various protamine degradation products.

FIG. 12 is a view showing the results of observation of a tongue tissue section by an optical microscope when a protamine hydrolysate and Hinokitiol are used in combination.

### DESCRIPTION OF EMBODIMENTS

The present inventors have found that, when a protamine hydrolysate obtained by hydrolyzing protamine and a terpene alcohol were used in combination for solving the problem of naturally occurring ingredients described above, an antifungal activity was enhanced remarkably and the enhanced effect was a synergistic effect exceeding an additive effect caused by the simple combined use, leading to the present invention.

Though it has been already known that each of a protamine hydrolysate and a terpene alcohol exhibits an anti-Candida effect when used singly, a finding that an antifungal activity was enhanced synergistically when these are used in combination has not been reported heretofore. In general, the definition of a synergistic effect when a microorganism is used in combination with an antibacterial substance indicates a case showing a significantly larger effect than the sum of individual effects of two agents. One criterion for evaluation on the presence or absence of a synergistic effect is FIC index (fractional inhibitory concentration index) determined from MIC values of two agents. FIC index can be determined by the following formula (1).

Method of determining FIC index

FIC index=A1/A0+B1/B0 (1)
A0: MIC when agent A is used alone
A1: MIC of agent A when agent A and agent B are used in combination
B0: MIC when agent B is used alone
B1: MIC of agent B when agent A and agent B are used in combination Evaluation of synergistic effect by FIC index
FIC≦0.5: synergistic effect
0.5<FIC≦1.0: additive effect
1.0<FIC≦2.0: no relation
2.0<FIC: antagonistic effect

Further, the present inventors have found that the synergistic effect for improving an antifungal activity by the combined use of a protamine hydrolysate and a terpene alcohol was manifested not only in a conventional in vitro evaluation system, but also in a biological model using oral candidiasis model mice, and the present inventors have completed the present invention based thereon. That is, it has been found that, when a protamine hydrolysate and a terpene alcohol were orally administered in combination, formation of white moss on mouse tongue was suppressed significantly, and an antifungal activity was improved synergistically, based on a phenomenon that white moss (candida fungal hyphae) was observed on tongue when oral candidiasis was allowed to occur in the mouse. Additionally, the present inventors have confirmed that a protamine hydrolysate had an antibacterial activity on fungi of the genus Malassezia.

Essential oils as a candidate material to be used in combination with a protamine hydrolysate were first screened in the in vitro evaluation system to investigate the synergistic effect described above. As a result, it was confirmed that an antifungal activity was improved synergistically by using a protamine hydrolysate in combination with a terpene alcohol such as Hinokitiol, terpinen-4-ol, geraniol, menthol, etc. In contrast, improvement of an antifungal activity was not confirmed in the the combined use with essential oils such as cinnamaldehyde and capric acid, which are not classified in terpene alcohols. Then, the the combined use of a protamine hydrolysate and Hinokitiol was evaluated in a biological model using an oral candidiasis model mouse. As a result, remarkable improvement of tongue score owing to a synergistic effect was recognized and maintenance of the synergistic activity under in vivo high salt concentration was confirmed. As described above, the present inventors has considered that the the combined use of a protamine hydrolysate and a terpene alcohol has inventive step in practical aspect over conventional technologies based on the antifungal activity improved at the same dosage or by far lower dosage, in comparison with the used amounts by using the conventional peptide compositions or the conventional essential oils. The present inventors have reached the present invention from the above consideration.

Next, the present invention will be described in detail.

Protamine as a raw material of a protamine hydrolysate is a strongly basic protein present in the form of a nucleoprotamine bonded to deoxyribonucleic acid in sperm nucleus of fish such as salmon, herring, trout, etc., and is called, for example, salmine (salmon), clupeine (herring), etc. depending on a difference of the raw material, and, although the structure varies slightly each other, any protamine can be used.

For hydrolysis of protamine, an acid, an alkali or a proteolytic enzyme can be used, alternatively. Decomposition by a combination thereof can be utilized, but use of a proteolytic enzyme is desirable. More specifically, the following procedures are used.

In the case of hydrolysis with an acid or an alkali, a protamine is heated under condition so that a peptide having an intended size (length) is obtained, in the presence of a strong acid or a strong alkali. The products are neutralized, and, thus, a peptide having an intended size (length) can be obtained.

Deionized water is added to a protamine, and sodium hydroxide or hydrochloric acid is added, to adjust pH to a value at which an enzymatic activity is obtained, preferably to optimum pH. The reaction solution is heated at a temperature wherein an enzymatic activity is obtained, preferably heated at the optimum temperature of an enzyme, then, an enzyme is added, and an enzymatic reaction is conducted while stirring. After completion of the reaction, the reaction liquid is heated at 80 to 100° C. and thermally deactivated for 5 to 60 minutes and pH is adjusted to neutral region, then, the reaction liquid is freeze-dried, thus, a protamine hydrolysate can be obtained. It is preferable that the hydrolysis enzymatic reaction is conducted until an arginine-rich peptide consisting of about 5 to 14 amino acid residues is obtained, and stopped by thermal deactivation of the enzyme.

Ingredients contained in the protamine hydrolysate obtained as described above can be used as an active ingredient of an antifungal composition. Therefore, the protamine hydrolysate can be used in the forms listed below.

(1) A reaction liquid having pH adjusted after thermal deactivation of the enzyme described above

(2) A freeze-dried product of the reaction liquid described above

(3) A preparation obtained by removing an enzyme protein from the reaction liquid or the freeze-dried product described above.

(4) A preparation obtained by change the peptide to its salt form in the above reaction liquid, the above freeze-dried product or the above preparation.

The proteolytic enzyme which can be used for hydrolysis in the present invention includes, for example, enzymes produced by the genus Bacillus (for example, Bacillus subtilis, Bacillus thermoproteolyticus, Bacillus licheniformis, etc.), enzymes produced by the genus Aspergillus (for example, Aspergillus oryzae, Aspergillus niger, Aspergillus melleus, etc.), enzymes produced by the genus Rhizopus (for example, Rhizopus niveus, Rhizopus delemar, etc.), pepsin, pancreatin, papain, etc. These enzymes may be used each singly or two or more of them may be combined. The proteolytic enzyme is classified into an endopeptidase specifically recognizing and cutting an internal sequence of a protein and an exopeptidase cutting every 1 to 2 amino acid residues from the end. Therefore, it is possible to form various peptide chains by a combination of an endopeptidase and an exopeptidase, as needed. In the case of hydrolysis with an enzyme, an enzyme is added in an amount of 0.001 to 10% by mass with respect to a substrate, and the solution is adjusted to the optimum pH of the enzyme(s) to be used and hydrolysis is performed.

A peptide contained in a protamine hydrolysate can be converted, if necessary, into the form of a salt with an inorganic acid or an organic acid or a salt with an inorganic base or an organic base, and used as an active ingredient of an antifungal agent. The acid and the base can be selected depending on the uses of the salt, and, when the use in food products, cosmetics, pharmaceutical products, etc. are considered, pharmaceutically acceptable salts listed below are preferable. The acid addition salts include, for example, hydrochlorides, nitrates, sulfates, methane sulfonates, p-toluene sulfonates, and further, salts with dicarboxylic acids such as oxalic acid, malonic acid, succinic acid, maleic acid, fumaric acid, etc., further, salts with monocarboxylic acids such as acetic acid, propionic acid, butyric acid, etc. The inorganic bases suitable for formation of a salt of a peptide compound obtained in the present invention include, for example, hydroxides, carbonates, bicarbonates, etc. of ammonium, sodium, lithium, calcium, magnesium, aluminum, etc. The salts with an organic base include, for example, mono-, di- and tri-alkylamine salts such as methylamine, dimethylamine and triethylamine, mono-, di- and tri-hydroxyalkylamine salts, guanidine salts, N-methylglucosamine salts, etc. A protamine hydrolysate having a peptide component converted into the form of a salt is also included in the protamine hydrolysate according to the present invention.

The composition having an antifungal activity according to the present invention, that is, the antifungal composition comprises a protamine hydrolysate and a terpene alcohol as the active ingredient, and it is preferable that the active ingredient consists of a protamine hydrolysate and a terpene alcohol.

The peptide having an antifungal activity obtained by hydrolysis of a protamine includes peptides classified into the following (1) to (6). Therefore, the protamine hydrolysate to be combined with a terpene alcohol is preferably one containing at least one peptide compound (namely, peptide or its salt) selected from the following groups (1) to (6).

(1) A peptide consists of an amino-acid sequence of SEQ ID No. 1 represented by Ile Arg Arg Arg Arg Pro Arg Arg, and a salt thereof.

(2) A peptide consists of an amino-acid sequence of SEQ ID No. 2 represented by Ser Arg Arg Arg Arg Arg Arg Gly Gly Arg Arg Arg Arg, and a salt thereof.

(3) A peptide consists of an amino-acid sequence of SEQ ID No. 3 represented by Val Ser Arg Arg Arg Arg Arg Arg Gly Gly Arg Arg Arg Arg, and a salt thereof.

(4) A peptide consists an amino-acid sequence obtained by deleting 1 to 6 amino acids in an amino-acid sequence of SEQ ID No. 1, or a salt thereof.

(5) A peptide consists of an amino-acid sequence obtained by deleting 1 to 4 amino acids in an amino-acid sequence of SEQ ID No. 3, or a salt thereof.

(6) A peptide consists of a deleted amino-acid sequence of SEQ ID No. 3 represented by Arg Arg Arg Arg Arg Arg Gly Gly Arg Arg Arg Arg (SEQ ID No. 4), Arg Arg Arg Arg Arg Gly Gly Arg Arg Arg Arg (SEQ ID No. 5) or Arg Arg Arg Arg Gly Gly Arg Arg Arg Arg (SEQ ID No. 6), or a salt thereof.

The protamine hydrolysate is more preferably one containing at least one peptide compound selected from the above-described groups (1) to (3). A protamine hydrolysate containing at least one peptide compound selected from the above-described group (1), at least one peptide compound selected from the above-described group (2) and at least one peptide compound selected from the above-described group (3) is further preferable.

Two or more of the protamine hydrolysates having an antifungal activity and having different formulations can be mixed and used.

Next, the terpene alcohol used in the present invention is preferably a mono-terpene alcohol having 10 carbon atoms in one molecule or a sesqui-terpene alcohol having 15 carbon atoms in one molecule. There are other essential oils and essential oil components having an antifungal activity, but all of them have side effects reported and have a problem of safety. For example, each of cinnamon bark oil and its constituent, cinnamaldehyde, lemongrass oil and its constituent, citral, time oil and its constituent, thymol, and oregano oil and its constituent, carvacrol, has strong dermal irridation. Further, although rosemary oil and its constituent, camphor, spearmint oil and its constituent, carvone, etc. have an antifungal activity, an irritation action to the central nerve system was reported regarding any one of them. In contrast, terpene alcohols such as geraniol, linalool and nerolidol cause no side effect which should be attended particularly, and have high safeness. Further, they are most suitable for the combined use with a protamine hydrolysate since they have a high antifungal activity as described above.

Plant essential oils and components thereof used in the present invention are as described below. Hinoki oil and its major constituent, Hinokitiol, tea tree oil and its major constituent, terpinen-4-ol, palmarosa oil and its major constituent, geraniol, peppermint oil and its major constituent, menthol and its derivative, lavender oil and its major constituent, linalool, patchouli oil and its major constituent, patchouli alcohol, lindera umbellata oil and its accessory constituent, nerolidol. Menthol derivatives include menthyl lactate, menthoxy propanediol, menthyl hydroxybutyrate, menthoxyfuran, menthylglucoside, etc. Further, any one of plant essential oils such as rosewood oil, rose geranium oil, lemon eucalyptus oil, peppermint oil and neroli oil can be also used by expecting the same effects.

As the mono-terpene alcohol, at least one selected from the group consisting of Hinokitiol, geraniol, terpinen-4-ol, menthol, menthol derivative and linalool is preferably used. It is further preferable that the mono-terpene alcohol is Hinokitiol, geraniol, terpinen-4-ol, menthol, menthol derivative or linalool.

As to sesqui-terpene alcohol, patchouli alcohol and/or nerolidol is/are preferable.

The antifungal composition according to the present invention can be obtained by dissolving or suspending a bulk powder (for example, freeze-dried product) of a protamine hydrolysate directly into an essential oil or essential oil component. The composition thus obtained can be administered as an antifungal agent, however, ointment or liniment such as cream and gel, or external preparations such as a spray product can be easily used, in practical aspect. The antifungal composition according to the present invention can contain at least one of an excipient, a diluent, a carrier, etc. depending on its administration form.

The essential oil or essential oil component can be prepared in the form of a completely clear liquid by using a suitable solvent such as ethanol and propylene glycol. The essential oil or essential oil component can also be suspended in water by using an aqueous gel such as Gel Nature or an essential oil dispersant such as a solubilizer. When used privately or in home, it is practical that the essential oil or essential oil component is previously diluted with a carrier oil and the resultant liquid is administered, in view of irritation and economy. The carrier oil for this purpose includes plant oils such furnace oil, jojoba oil, sweet almond oil, neem oil, St. John's Wort oil, etc. Further, if an antifungal composition is added to a foot bath of hot water, the same effect is expected.

The antifungal composition according to the present invention comprising both a protamine hydrolysate and a terpene alcohol as an active ingredient exhibits a high antifungal activity against at least one of fungus, and particularly, fungi of the genus Candida and the genus Malassezia as a disease-causing fungi of fungal infection diseases are preferable as the target, and additionally, for example, the genus Cryptococcus, the genus Aspergillus, the genus Trichophyton, the genus Coccidioides, etc. can be adopted as the target. The antifungal activity according to the present invention can be confirmed by using a fungal strain belonging to these genera, according to an ordinary method, for example, according to a method mentioned in the examples described later.

Since a peptide compound is supposed to scarcely generate a drug-resistant strain, a protamine hydrolysate is suitably used as the major constituent of a fungus disease preventing agent. Further, since safeness is confirmed for a protamine hydrolysate and a terpene alcohol, these are suitable as an active ingredient of an antifungal composition. The antifungal composition according to the present invention can be contained in food products, pharmaceutical products, quasi-pharmaceutical products and cosmetics as an active ingredient of an antifungal agent, to obtain effects such as prophylaxis, alleviation, treatment, etc. of fungal infection diseases. Further, the antifungal composition of the present invention can be used as an active ingredient of a composition for prophylaxis and/or treatment of fungal infection diseases. The antifungal composition of the present invention can be used as an active ingredient in a method of prophylaxis and/or treatment of fungal infection diseases, by comprising administration of an effective amount of the antifungal composition according to the present invention to the subject of prophylaxis and/or treatment of fungal infection diseases.

The formulation of the antifungal composition is not particularly restricted. For example, typical formulations of internal preparations or external preparations include ointment, liquid, suspension, emulsion, aerosol, foam, granule, powder, tablet, capsule and spray. The formulation can also be a freeze-dried material or a granule, which is dissolved in physiological saline or suitable buffer fluid (for example, PBS) to prepare a liquid drug directly before its use as an injection, etc. The production processes to prepare the various drug formulations (compositions) per se can be conducted according to the conventional known processes from the materials comprising a protamine hydrolysate and a terpene alcohol (active ingredients), and various carriers (secondary ingredients). Since such production processes do not characterize the present invention, the detailed explanation thereof is omitted. The detailed information sources regarding the prescriptions include, for example, Comprehensive Medicinal Chemistry, edited by Corwin Hansch, published by Pergamon Press (1990).

The contents of a protamine hydrolysate having an antifungal activity and a terpene alcohol as an active ingredient in an antifungal composition can be selected from a range in which the synergistic effect of them targeted in the present invention is obtained, depending on its use. Further, the blending amounts of these active ingredients can be selected likewise from a range so as to obtain the synergistic effect can be obtained in pharmaceutical products, quasi-pharmaceutical products, food products and cosmetics, as the objects to which the function as an antifungal agent is provided.

The antifungal composition provided by the present invention can be used according to the method and the dosage corresponding to its formulation and objects. For example, the antifungal composition provided by the present invention can be administered in the formulation of a liquid preparation by intravenous, intramuscular, subcutaneous, intradermal or intraperitoneal injection or colonic irrigation. The composition in a solid formulation such as tablet, etc. can be administered orally. A liquid preparation containing a relatively large amount of a protamine hydrolysate (for example, 1 to 100 mg/ml in terms of the amount of a peptide compound) may be sprayed directly on the surfaces of the targets, or the surfaces of the targets may be wiped with gauze, cloth or paper wetted with the liquid preparation(s), when the antifungal composition is used for disinfection (sterilization) of the surface of skin, the inside of the oral cavity, the inside of the vagina or the surface of a sanitary ware; or for food preservation. The above applications are only examples, and the same formulations and the same methods for the uses can be applied as those for the conventional peptide antibiotics and the agricultural compositions, the quasi-pharmaceutical products, etc. which contain a peptide as a component. For example, for cancer patients and AIDS patients undergoing radiation therapy, prophylaxis and treatment of infection diseases due to microorganisms are important concerns. The antifungal composition herein disclosed can exhibit a high antifungal action to fungi (for example, Candida fungi) as a cause of an infection disease. For this reason, the antifungal composition of the present invention is useful as the major constituent of an antifungal agent.

The means according to the present invention for performing prevention, alleviation, treatment, etc. include the uses in a food product, a functional food product, a pharmaceutical product, a quasi-pharmaceutical product or a cosmetic, such as the addition of the antifungal composition to gums, granular confectioneries, tablet cakes, films, sprays, health assistant food products, foods for specified health uses, implements for oral or dental hygiene, denture cleaning agents and denture stabilizer compositions; and various formulations such as mouthwashes, mouth cleaning agents, medical tooth powders, lotions, creams, powders, emulsions, sprays, plasters etc.

### EXAMPLES

Next, the preferable embodiments for carrying out the present invention will be described in detail by showing the examples, but the present invention is not limited to the following examples.

The following fungal strains are available from the following institutes. The fungal strains for testing an antifungal activity are not limited to the strains listed below, and generally used strains can be used.

-Candida albicans NBRC1594

Independent administrative agency National Institute of Technology and Evaluation, Biotechnology Center

(Contact name: Biological Resource Utilization Promotion Division)

Address: 2-5-8 Kazusakamatari Kisarazu city, Chiba prefecture 292-0818, Japan (http://www.nite.go.jp/nbrc/cultures/)

-Candida albicans TIMM1768 and Malassezia Fur Fur TIMM10020

Teikyo University Medical Fungus Research Center

Address: 359 Otsuka Hachioji city, Tokyo 192-0395, Japan (http://www.teikyo-u.ac.jp/timm/research/r01.html?page=research)

### Example 1

### (Preparation of Protamine Hydrolysate by Enzymatic Degradation of Protamine)

To 50 g of protamine derived from milts of Chum salmon (Oncorhynchus keta) (Proserve; manufactured by Maruha Nichiro Corporation) was added 80 mL of deionized water, and sodium hydroxide was added to the resultant mixture to adjust pH to 8.0. The solution was heated to 65° C., then, 1.5 mg of thermolysin (manufactured by Nacalai Tesque Inc., derived from Bacillus thermoproteolyticus) was added, and an enzymatic reaction was conducted while stirring for 2 hours. After completion of the reaction, the reaction liquid was heated to 95° C. and thermally deactivated for 30 minutes and pH was adjusted to 8.5. Thereafter, the reaction liquid was freeze-dried, to obtain a protamine hydrolysate.

### Example 2

### (Isolation of Active Fraction of Protamine Hydrolysate and Analysis of Active Peptide)

The protamine hydrolysate prepared according to Example 1 was prepared into a 50000 ppm solution using deionized water, then, each fraction was isolated under the following separation conditions using HPLC. The anti-candida activity of the fractions were evaluated using Candida albicans NBRC1594, and two active fractions were obtained. Further, these active fractions were subjected for LCMS-IT-TOF analysis, and the structures of the following three peptides were determined.

(1) Ile Arg Arg Arg Arg Pro Arg Arg (8 residues; molecular weight: 1164.7541)

(2) Ser Arg Arg Arg Arg Arg Arg Gly Gly Arg Arg Arg Arg (13 residues; molecular weight: 1780.0966)

(3) Val Ser Arg Arg Arg Arg Arg Arg Gly Gly Arg Arg Arg Arg (14 residues; molecular weight: 1879.1650).

### <HPLC Separation Condition>

HPLC system: manufactured by Shimadzu Corporation, Prominence series (system controller: CBM-20A, auto sampler: SIL-10AF, solution sending pump: LC-8A×2 pumps, column oven: CTO-20A, PDA detector: SPD-M20A (measuring wavelength: 190 to 400 nm))
flow rate: 1.0 mL/min
mobile phase A: 0.1% trifluoroacetic acid
mobile phase B: acetonitrile
column: Inertsil ODS-2 5 μm 4.6 mm×250 mm (manufactured by GL Science Inc.)
gradient: linear gradient from 0% by volume of liquid B to 15% by volume of liquid B ranging from 0 to 60 minutes
detection wavelength: 214 nm.

### <LCMS-IT-TOF Analysis Condition>

HPLC system: manufactured by Shimadzu Corporation, Prominence series (system controller: CBM-20A, auto sampler: SIL-20A, solution sending pump: LC-20AB Binary pump, column oven: CTO-20A, PDA detector: SPD-M20A (measuring wavelength: 190 to 400 nm))
flow rate: 0.2 mL/min
mobile phase A: 0.1% formic acid (manufactured by Kanto Chemical Co., Inc., for LC-MS)
mobile phase B: acetonitrile (manufactured by Kanto Chemical Co., Inc., for LC-MS)
column: Atlantis HILIC Silica, 3 μm Column (manufactured by Waters)
gradient: linear gradient from 60% by volume of liquid B to 0% by volume of liquid B ranging from 0 to 30 minutes
injection volume: 10 μL
IT-TOFMS system: manufactured by Shimadzu Corporation, LCMS-IT-TOF (ionization mode: ESI−, atomized gas flow rate: 1.5 L/min, Drying gas pressure: 0.15 Mpa, applied voltage: 4.5 kV, CDL temperature: 200° C., BH temperature: 200° C., measurement range MS: m/z 500 to 2,000).

### Example 3

### (In Vitro Screening of Material Performing Synergistic Effect)

### Strain

Candida albicans TIMM1768

### Test Material

Hinokitiol (manufactured by Kiseitech Co., Ltd.), terpinen-4-ol (manufactured by Tokyo Chemical Industry, Co., Ltd.), geraniol (manufactured by Kanto Chemical Co., Inc.), menthol (manufactured by Wako Pure Chemical Industries, Ltd.), cinnamaldehyde (manufactured by Wako Pure Chemical Industries, Ltd.) and capric acid (manufactured by Wako Pure Chemical Industries, Ltd.) were individually dissolved in RPMI medium so that the concentration in a test material was 0.008, 0.04, 0.2, 1 mg/mL, and the protamine hydrolysate prepared according to Example 1 was dissolved so that the concentration of the protamine hydrolysate in a test material was 0, 0.125, 2.0 mg/mL, and the solutions were subjected to the test. Further, 0.002, 0.008, 0.031, 0.125, 0.5, 2, 8, 32 mg/mL solutions using the protamine hydrolysate singly were subjected to the test. Evaluation method

C. albicans TIMM1768 strain was added to a 96 well microplate each in an amount of 200 μL so that 2.5×10⁵ cells/well, and cultured for 2.5 to 3.5 hours at 37° C. under 5% CO₂. After cultivation, each medium was removed under suction, and each test material was added in an amount of 200 μL/well. After allowing to stand still at 37° C. for 10 minutes, each sample solution was removed under suction, and RPMI medium was added in an amount of 200 μL/well, and cultured for 2.5 hours at 37° C. under 5% CO₂. After cultivation, each sample was stained with crystal violet and the absorbance at 620 nm was measured. The antibacterial activity was expressed in terms of the relative value of each experimental section when the blank absorbance was 1.

### Result

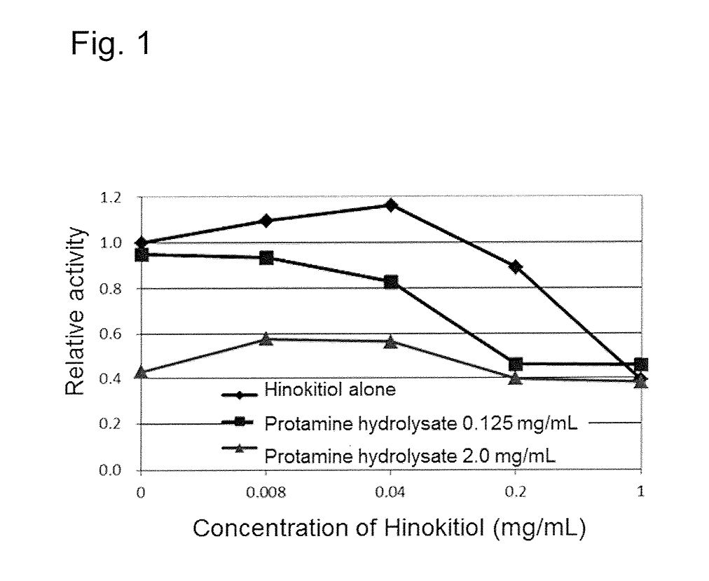

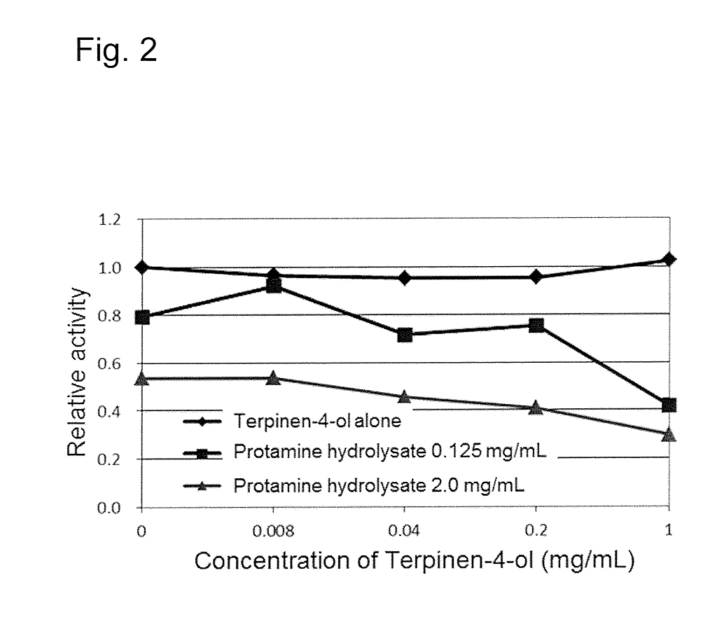

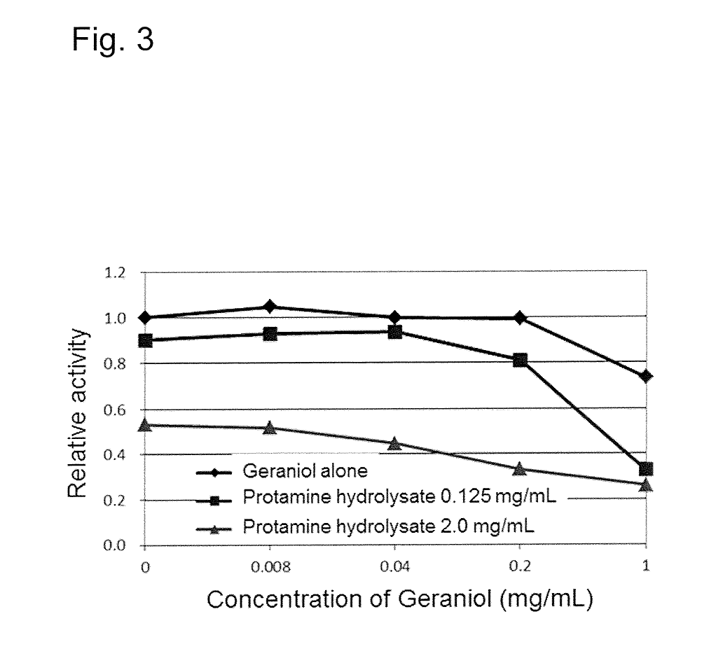

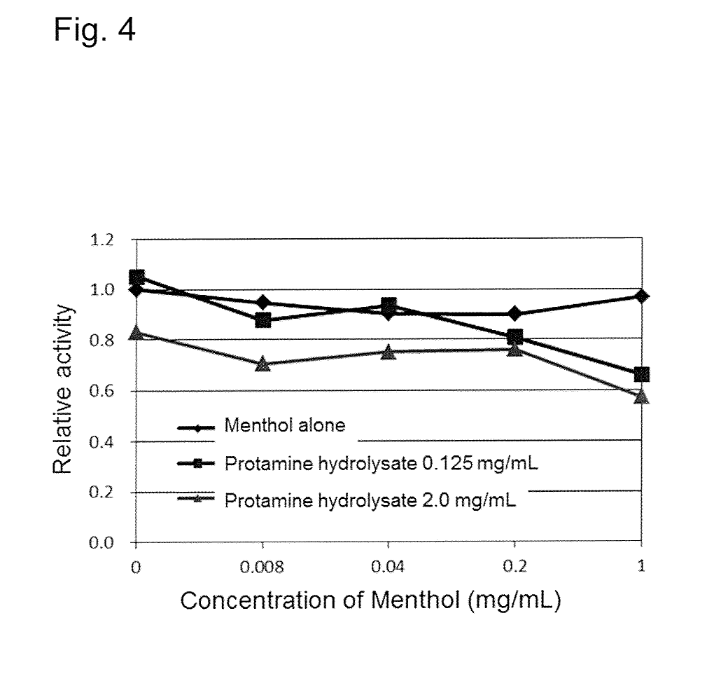

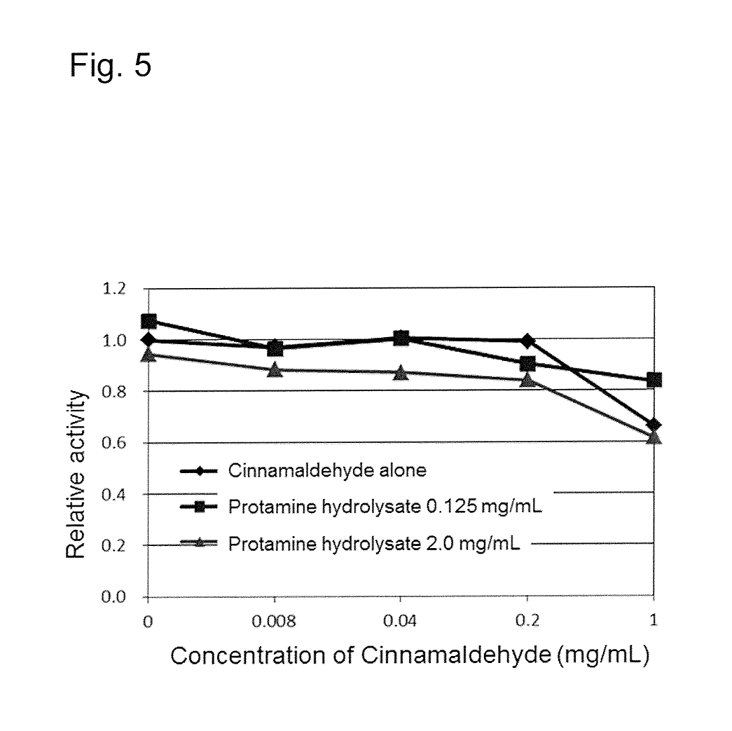

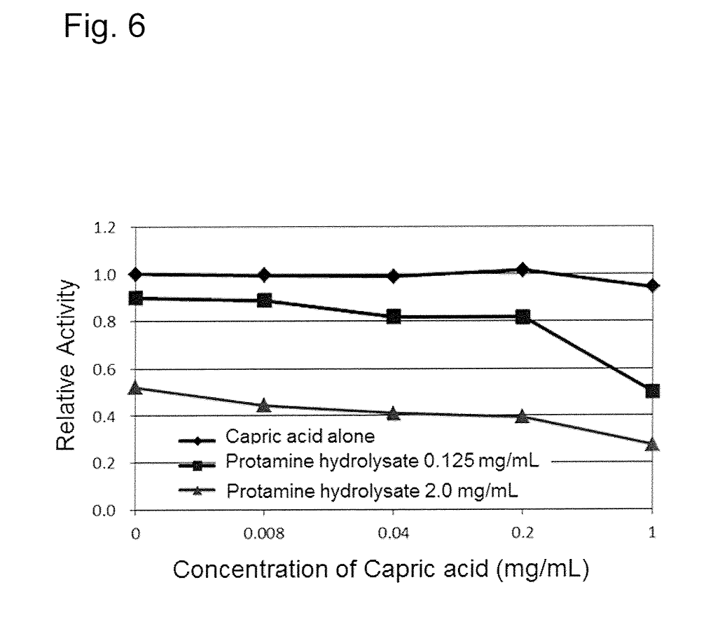

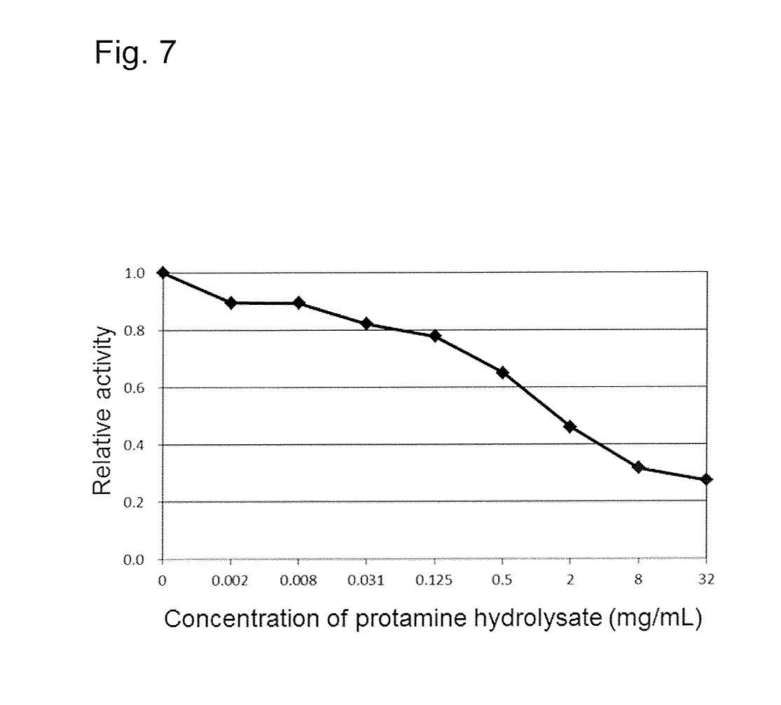

The results of in vitro evaluation when using a protamine hydrolysate and terpene alcohols in combination are shown in FIGS. 1 to 6, and the result of in vitro evaluation when using a protamine hydrolysate singly is shown in FIG. 7. In FIGS. 1 to 6, ♦ represents the result of single use of terpene alcohols, ▪ represents the result of the combined use with a protamine hydrolysate 0.125 mg/ml, and ▴ represents the result of the combined use with a protamine hydrolysate 2.0 mg/ml, respectively.

Hinokitiol showed an antibacterial activity even in the single use, and it was believed that a synergistic effect of improving activity by the combined use with a protamine hydrolysate was recognized. Although terpinen-4-ol, geraniol and menthol showed low activity when they were used singly, the activity was slightly improved by the combined use with a protamine hydrolysate, therefore, the synergistic effect was believed to exist. Cinnamaldehyde and capric acid showed little antibacterial activity when they were used singly and the synergistic effect by the combined use with a protamine hydrolysate was not recognized. Although it is known that capric acid shows a strong antifungal activity, it was believed that capric acid could not exhibit sufficient antifungal action since the contact time of the drug with the fungus was set at as short as 10 minutes because this in vitro evaluation system mimicked in vivo.

Based on the results described above, it was confirmed that a protamine hydrolysate shows a synergistic effect with terpene alcohols such as Hinokitiol, terpinen-4-ol, geraniol, etc. among the essential oils.

### Example 4

### (Confirmation of Effect in In Vivo Evaluation System)

### Strain

Candida albicans TIMM1768

### Laboratory Animal

ICR mouse, female, 6-week old (manufactured by Charles River Laboratories Japan, Inc.)

### Test Material

Protamine hydrolysate prepared according to Example 1

Hinokitiol (manufactured by Kiseitech Co., Ltd.)

### Group Constitution

Negative control (1% methylcellulose), six mice

Protamine hydrolysate 25 mg/mL (1% Tween 80), six mice

Hinokitiol 0.4 mg/mL (1% Tween 80), six mice

Hinokitiol 2 mg/mL (1% Tween 80), six mice

Protamine hydrolysate 25 mg/mL+Hinokitiol 0.4 mg/mL (1% Tween 80), six mice

Protamine hydrolysate 25 mg/mL+Hinokitiol 2 mg/mL (1% Tween 80), six mice Evaluation method

The day before injection of C. albicans, 100 mg/kg of an immunosuppressive agent (prednisolone) was subcutaneously injected. For preventing injection by other fungi than C. albicans, chlortetracycline hydrochloride was contained in tap water at 15 mg/mL, and the animal was allowed to intake water freely. On the day of injection, 14.4 mg/kg of chlorpromazine hydrochloride was intramuscularly injected, then, C. albicans was diluted with FBS-RPMI liquid medium so that 2×10⁸ cells/mL, and the diluted fungus liquid was applied to the mouse oral cavity by a cotton-tipped stick. Then, 3, 24, 27 hours after infection, 50 μL of each test material was administered using an oral probe (sonde). Then, 48 hours after infection, the mouse was euthanized, and the tongue condition was scored. The tongue score was 0 point when white moss was not recognized visually at all, 1 point when white moss was confirmed in regions of 20% or less of the tongue, 2 points when 21 to 90%, 3 points when 91% or more, and 4 points when 91% or more and laminated white moss was confirmed.

### Result

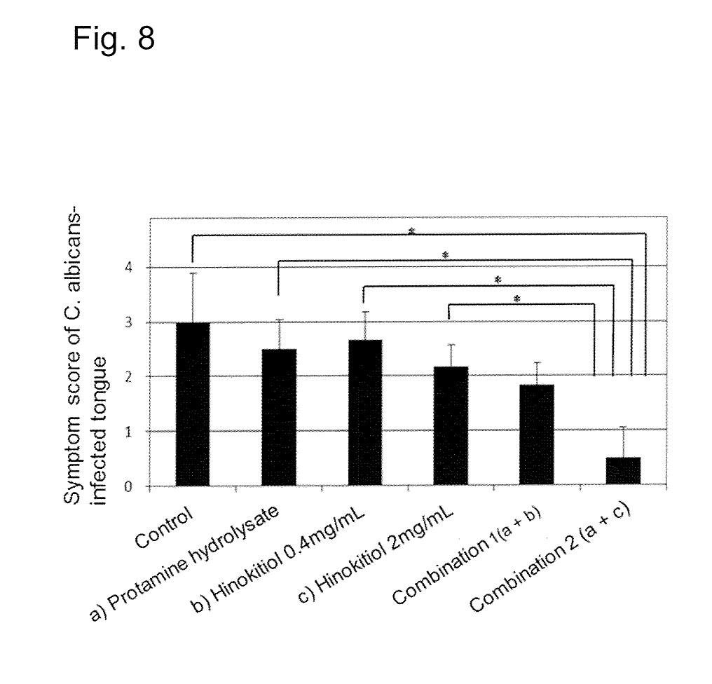

The result of in vivo evaluation by the combined use of a protamine hydrolysate and Hinokitiol is shown in FIG. 8. A significant improvement of the symptone score of C. albicans-infected tongue was confirmed by a combination of a protamine hydrolysate 25 mg/mL and Hinokitiol 2.0 mg/mL, the score was remarkably improved as compared with a case of single administration of each material, thus, an improvement of an antifungal activity by a synergistic effect was confirmed. An action mechanism of adhering to the surface of a fungus body and showing bacteriostasis at low concentration was suggested concerning a protamine hydrolysate, while inhibition of membrane functions and respiratory suppressing action were reported concerning Hinokitiol. Regarding such difference of their mechanisms, it was considered that the combined use of a protamine hydrolysate and Hinokitiol did not cause a simple additive effect but caused a synergistic effect to enhance the antibacterial activity.

### Reference Example 1

### (Confirmation of Effect for Malassezia Fungus)

### Strain

Malassezia fur fur TIMM10020

### Evaluation Method

Malassezia fur fur cultured in MLNA medium was adjusted to McFarland 1.0 by physiological saline, and 100 μL of the prepared liquid and 50 μL of olive oil were spread on MLNA agar plate. At the center of a petri dish, 50 μL of a test sample (a protamine hydrolysate prepared to 40 mg/mL with sterile water) was dropped on an 8 mm paper disk for antibiotic assay, and cultured for 7 days at 32° C.

### Result

An inhibition ring of 14 mm×14 mm (paper disk had a thickness of 8 mm) was observed around the paper disk on which 50 μL of the protamine hydrolysate (40 mg/mL) had been dropped. It was confirmed that the protamine hydrolysate had an antibacterial effect to the Malassezia fungus, since an inhibition ring was not observed around sterile water as a control.

### Example 5

(Detailed Analysis with Respect to Use in Combination with Hinokitiol)

### Strain

Candida albicans TIMM1768

### Test Material

Hinokitiol (manufactured by Kiseitech Co., Ltd.) was dissolved in RPMI medium so that the concentration in each test material was 0, 0.004, 0.008, 0.016, 0.031, 0.063, 0.125, 0.25, 0.5, or 1 μg/mL, and the protamine hydrolysate prepared according to Example 1 was dissolved so that the concentration of the protamine hydrolysate in each test material was 0, 0.156, 0.313, 0.625, 1.25, 2.5, 5, or 10 μg/mL, and they were subjected to the test.

### Evaluation Method

C. albicans TIMM1768 strain was diluted with RPMI medium so that 5.0×10³ cells/m L, then, added to a 96 well microplate each in an amount of 100 μL. The protamine hydrolysate solution was added each in an amount of 50 μL/well, then, Hinokitiol was added each in an amount of 50 μL/well. After cultivation for 16 hours at 37° C. under 5% CO₂, each sample was stained with crystal violet and the absorbance at 620 nm was measured. The inhibitory activity was calculated from the relative value of each experimental section when the absorbance of a negative control (no addition of protamine hydrolysate and Hinokitiol) was 1. Further, FIC values were also calculated together at the concentrations of Hinokitiol (manufactured by Kiseitech Co., Ltd.) and a protamine hydrolysate “Pro” shown in Table 1, respectively.

### Result

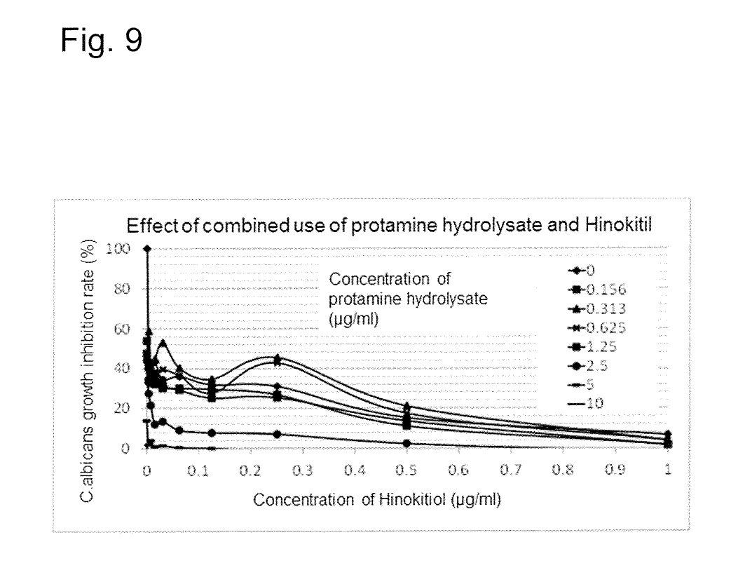

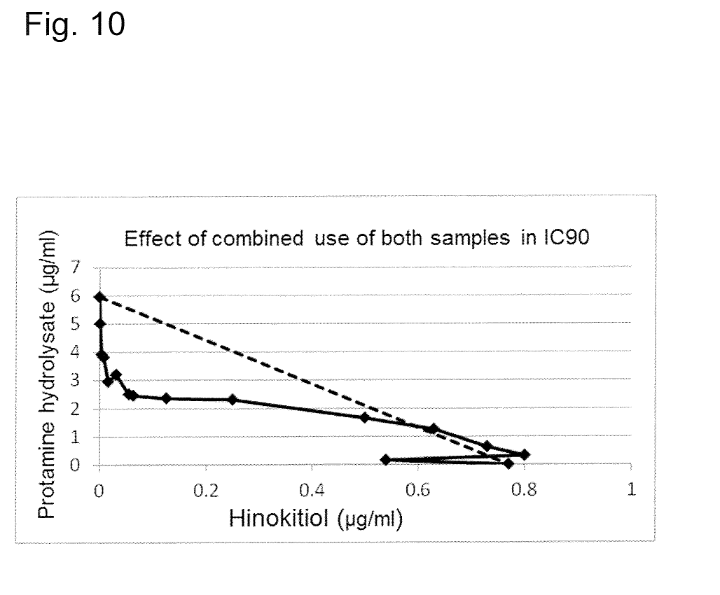

The results of in vitro evaluation when using a protamine hydrolysate and Hinokitiol in combination are shown in FIG. 9 and FIG. 10. The results of the calculated FIC index values are shown in FIG. 1.

TABLE 1
IC90

| hinokitiol | Pro | FIC |
|---|---|---|
| 0 | 5.95 | 1.00 |
| 0.001 | 5 | 0.84 |
| 0.004 | 3.9 | 0.66 |
| 0.008 | 3.8 | 0.65 |
| 0.016 | 2.95 | 0.52 |
| 0.031 | 3.2 | 0.58 |
| 0.055 | 2.5 | 0.49 |
| 0.063 | 2.45 | 0.49 |
| 0.125 | 2.35 | 0.56 |
| 0.25 | 2.3 | 0.71 |
| 0.5 | 1.65 | 0.93 |
| 0.63 | 1.25 | 1.03 |
| 0.73 | 0.625 | 1.05 |
| 0.8 | 0.313 | 1.09 |
| 0.54 | 0.156 | 0.73 |
| 0.77 | 0 | 1.00 |

As a result, a synergistic effect of FIC≦0.5 was observed in the combination of a protamine hydrolysate and Hinokitiol at low concentration.

Based on the above-described results, it was confirmed that a protamine hydrolysate shows a synergistic effect with Hinokitiol.

### Example 6

(In Vitro Evaluation of Protamine Hydrolysate Derived from Milt of Other Fishs)

### Strain

Candida albicans TIMM1768

### Test Material

A protamine hydrolysate derived from each fish (pink salmon, herring) prepared according to Example 1 and a bromelain, a degradation product of a protamine derived from salmon milts were dissolved in RPMI medium so that the concentration of the protamine hydrolysate in each test material was 0.625, 1.25, 2.5, 5, 10, 20, 40, 80, or 160 μg/m L, and they were subjected to the test.

### Evaluation Method

C. albicans TIMM1768 strain was diluted with RPMI medium so that 5.0×10³ cells/m L, then, added to a 96 well microplate each in an amount of 100 μL. A protamine hydrolysate solution was added each in an amount of 50 μL/well, then, RPMI medium was added each in an amount of 50 μL/well. After cultivation for 16 hours at 37° C. under 5% CO₂, each sample was stained with crystal violet and the absorbance at 620 nm was measured. The inhibitory activity was calculated from the relative value of each experimental section when the absorbance of a negative control (no addition of protamine hydrolysate) was 1.

### Result

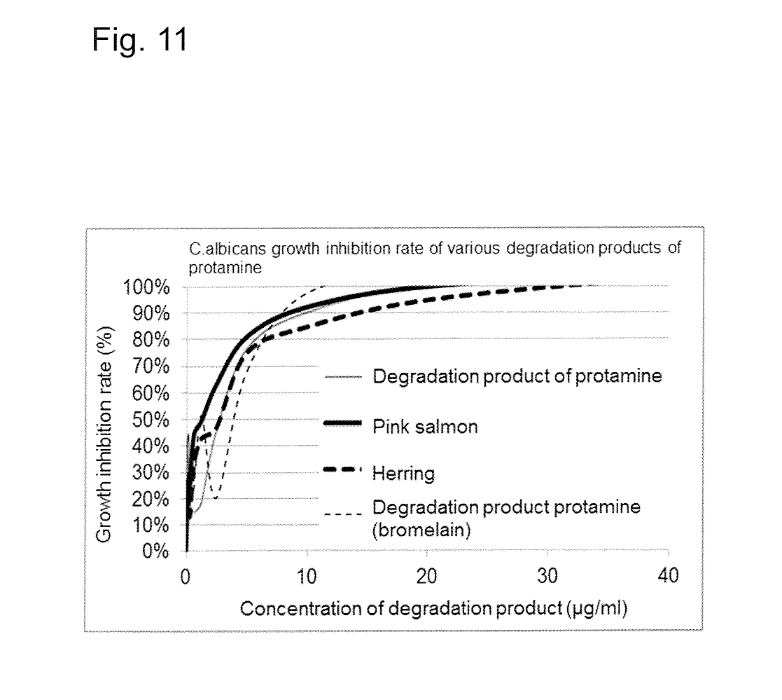

The results of in vitro evaluation of various protamine hydrolysates are shown in FIG. 11.

All test materials showed an activity equivalent to the protamine hydrolysate derived from salmon milt. This result shows versatility of the protamine degradation products for the kinds of fish.

### Example 7

### (Confirmation of Effect of the Combined Use in In Vivo Evaluation System)

### Strain, Laboratory Animal, Test Material, Group Constitution, Evaluation Method

The same strain, laboratory animal, test material, group constitution and evaluation method as in Example 4 were used.

From each mouse euthanized 48 hours after injection, a tong tissue section was made by an ordinary method, and stained, then, filmed by an optical microscope, to obtain a stained image.

### Result

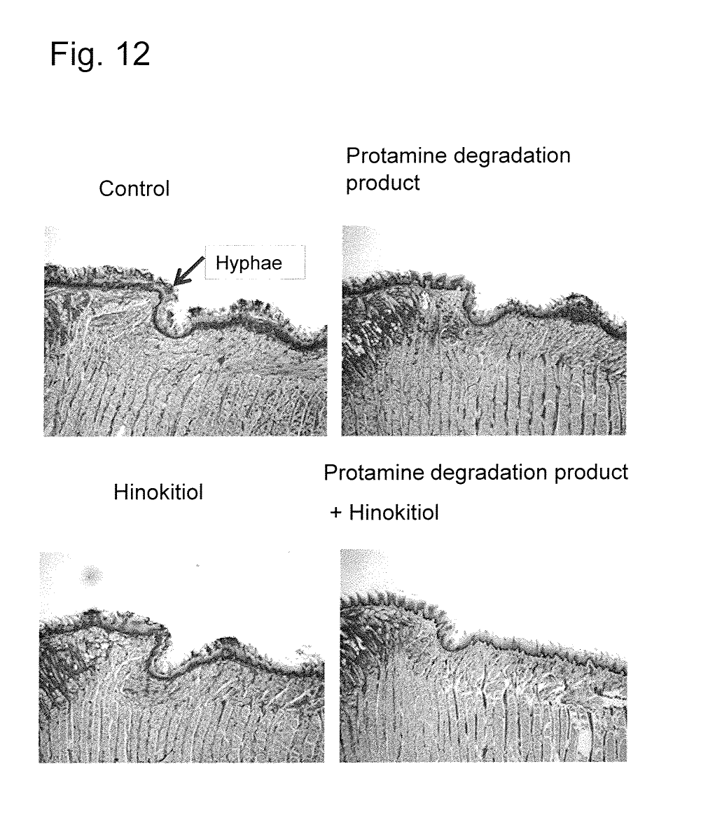

The result of observation of the tongue tissue when using a protamine hydrolysate and Hinokitiol in combination is shown in FIG. 12. Also in the tongue tissue image, fungal hyphae remained in mucosal membrane on the tongue surface when a protamine degradation product was used singly, while suppression of fungal hyphae was clearly confirmed in the experimental section of the combined use.

1. A composition having an antifungal activity, which comprises a protamine hydrolysate and a terpene alcohol, as an active ingredient.

2. The composition having an antifungal activity according to claim 1, wherein said protamine hydrolysate comprises at least one peptide compound comprising a peptide consisting of an amino acid sequence of any one of SEQ ID Nos. 1 to 3 and salts thereof:
SEQ ID No. 1: Ile Arg Arg Arg Arg Pro Arg Arg;,
SEQ ID No. 2: Ser Arg Arg Arg Arg Arg Arg Gly Gly
Arg Arg Arg Arg;
and
SEQ ID No. 3: Val Ser Arg Arg Arg Arg Arg Arg Gly
Gly Arg Arg Arg Arg.

3. The composition having an antifungal activity according to claim 1, wherein said terpene alcohol is selected from mono-terpene alcohols or sesqui-terpene alcohols.

4. The composition having an antifungal activity according to claim 3, wherein said mono-terpene alcohol is Hinokitiol, geraniol, terpinen-4-ol, menthol, menthol derivative or linalool.

5. The composition having an antifungal activity according to claim 4, wherein said sesqui-terpene alcohol is a patchouli alcohol or nerolidol.

6. An antifungal agent comprising the composition having an antifungal activity according to claim 1.

7. A food comprising the composition having an antifungal activity according to claim 1.

8. A pharmaceutical product or a quasi-pharmaceutical product comprising the composition having an antifungal activity according to claim 1.

9. A cosmetic comprising the composition having an antifungal activity according to claim 1.

10. A method of using the composition having an antifungal activity according to claim 1 as an active ingredient for obtaining an antifungal activity in production of a food product or a cosmetic.

11. A method of using the composition having an antifungal activity according to claim 1 as an active ingredient for obtaining an antifungal activity in production of a pharmaceutical product or a quasi-pharmaceutical product.


SEQUENCE LISTING (6 sequences)

SEQ ID NO: 1 - protein, 8 aa, Oncorhynchus keta
IRRRRPRR

SEQ ID NO: 2 - protein, 13 aa, Oncorhynchus keta
SRRRRRRGGRRRR

SEQ ID NO: 3 - protein, 14 aa, Oncorhynchus keta
VSRRRRRRGGRRRR

SEQ ID NO: 4 - protein, 12 aa, Oncorhynchus keta
RRRRRRGGRRRR

SEQ ID NO: 5 - protein, 11 aa, Oncorhynchus keta
RRRRRGGRRRR

SEQ ID NO: 6 - protein, 10 aa, Oncorhynchus keta
RRRRGGRRRR